(19) United States
(12) Patent Application Publication
(10) Pub. No.: US 2010/0331608 A1
(43) Pub. Date: Dec. 30, 2010

(54) SURGICALLY IMPLANTABLE URETHRA PRESSURE CONTROL VALVE

(76) Inventor: Antoine Trubiano, Montreal (CA)

Correspondence Address: OGILVY RENAULT LLP, 1, Place Ville Marie, SUITE 2500, MONTREAL, QC, H3B 1R1, CA

(21) Appl. No.: 12/818,190

(22) Filed: Jun. 18, 2010

(30) Foreign Application Priority Data
Jun. 25, 2009 (CA) 2,670,554

(51) Int. Cl. A61F 2/02

(52) U.S. Cl. 600/30

(57) ABSTRACT

A surgically implantable urethra pressure control valve is described. It comprises a clamp having a pair of spaced-apart clamping arms. A urethra passage is defined between the clamping arms. At least one of the clamping arms is a displaceable clamping arm movable towards the other clamping arm to a predetermined position spaced from the other clamping arm a distance sufficient to pinch and close an inner passage of the urethra when disposed in the urethra passage whereby to arrest the flow of urine. An actuator is provided to effect the displacement of the displaceable clamping arm. The actuator may be a remote control to operate the valve or an implanted sphincter to apply a closing pressure upon the urethra.

### TECHNICAL FIELD

The present invention relates to a surgically implantable urethra pressure control valve and particularly to improvements thereof wherein the valve may be remotely operated or include a sphincter for operation by the user urging urine pressure thereagainst.

### BACKGROUND ART

In my co-pending U.S. application Ser. No. 11/775,259, entitled “Urethra Pressure Control Valve to Control Incontinence”, filed on Jul. 10, 2007, is described a surgical implantable urethra pressure control sphincter valve wherein an inflated balloon is retained captive inside a circumferential clamp which is disposed about the urethra in a patient's body whereby to close the urethra by applying a predetermined pressure thereto. The present invention relates to improvements in such pressure control sphincter valves.

Urinary incontinence is defined as the accidental leakage of urine through the urethra. Prostate problems and post radical prostatectomy urinary incontinence greatly affects a male's quality of life. The National Institute of Diabetes and Digestive and Kidney Diseases (NIDDK) has reported that urinary incontinence is a medical problem and that there are four forms of urinary incontinence. These are (1) temporary and reversible incontinence related to urinary track infection, constipation or delirium; (2) stress incontinence caused by weak pelvic and sphincter muscles; (3) urgent continence caused by damaged or iritatable nerves; and (4) overflow incontinence that results when an individual is unable to empty the bladder.

The urinary system, to do its job, muscles and nerves must work together to hold urine in the bladder and then release it at the right moment. A person develops the sphincter muscle control as a normal phenomenon associated with nerve signals. These muscles cause the bladder to squeeze and exude liquid therefrom.

The present invention is particularly concerned, but not exclusively, with a urethra pressure control adjustable valve which essentially replaces the prostate in men. The prostate is a male gland about the size and shape of a walnut that surrounds the urethra immediately below the bladder. To treat prostate cancer the prostate gland is usually surgically removed and this could cause problems to the muscles that control the bladder amongst other side effects. The loss of control by the bladder muscles will cause uncontrollable leakage. Various methods and devices have been developed to try and treat this problem. One such treatment is to insert a catheter through the urethra to drain the bladder. The catheter then leads to a bag in which the fluid from the bladder is collected. A major problem with these catheters is that they often develop infections and stone formation not to mention the discomfort of carrying and empting bag on a regular basis. They also require frequent disinfecting and cleaning. Cauterization is usually done by a doctor but a patient may be easily trained to effect the procedure himself. To do this, there is a need to learn sterile techniques to avoid urinary track infections.

A more recent technique is to use an artificial sphincter which is implanted adjacent the urethra below the bladder to keep the urethra closed until it is time to urinate. As reported in medical publications, this device can help people who have incontinence because of weak sphincter muscles or because of nerve damage that interferes with sphincter muscle function. It does not solve incontinence caused by uncontrolled bladder contraction. Artificial sphincters consist of a cup that fits around the urethra with a small balloon reservoir placed in the abdomen and a pump placed in the scrotum. The cup is filled with a liquid that makes it fit tightly around the urethra to squeeze the urethra to prevent urine from leaking. When it is time to urinate you squeeze the pump with the fingers to deflate the cup so that the liquid moves to the balloon reservoir from the cuff and urine can now flow through the urethra. When the bladder is emptied, the cup automatically refills within a time delay of about 2 to 5 minutes to keep the urethra tightly closed. This solution has not been found to work efficiently and requires interaction with the user to release the urine.

In recent years a new procedure has been developed to treat urinary incontinence. This new procedure comprises implanting a balloon which is connected to a conduit tube with the conduit tube remaining inside a person's body and the balloon is positioned adjacent the urethra whereby upon inflation of the balloon, through the scrotum, the urethra will be squeezed and hopefully close. The tube is provided with an inlet port positioned in the scrotum and through which a fluid is injected by a syringe, through the skin of the scrotum, whereby to inject a proper amount of fluid in the balloon to expand it to apply sufficient pressure against the urethra. This technique has also encountered various problems, and it has been reported that the success rate is no better than fifty percent (50%). A major problem with this technique is that the urethra is unstable and when pressure is applied against it the urethra will be displaced in an uncontrollable manner. The balloons are also unstable. This is why the efficiency rate has not been satisfactory. Usually there are two of these balloons that are implanted one on opposed sides of the urethra and sometimes offset from one another. Reference to U.S. Pat. Nos. 6,045,498 and 6,445,138 describes such implantable devices and their operation.

As reported in Medical News Today, Newsletter dated Oct. 24, 2006, these balloons are implanted beneath the bladder neck to increase its resistance. The novel difference with this device is the ability to adjust the tightness of the urethral occlusion by altering the amount to fluid in each balloon via a titanium port connector that can be accessed via a percutaneous injection in the scrotum. A study of this technique is also reported in the May 2006 issue of Urology. With this technique balloon adjustment is required to achieve continence and the average number of adjustments was 4.6, all of which were done in an out patient setting and in first six months after placement. A revision surgery was also required in four of twenty-three patients.

The above-mentioned technique appears to be on course to eventually resolve problems associated with balloon implants. However, there is still a need to resolve major problems with this technique such as the assurance that the implant will effectively engage the urethra and effect proper closure thereof by applying a pressure customized to the patient's needs depending on his degree of control to evacuate urine from the bladder. Another problem to be resolved is the implantation of the device itself about the urethra to effectively assure the proper function thereof prior to closing the incision.

It has also been reported by NIDDK that women experience incontinence twice as often as men. Pregnancy and childbirth, menopause, and the structure of the female urinary tract account for this difference. But both women and men can become incontinent from neurologic injury, birth defects, strokes, multiple sclerosis and physical problems associated with aging. Incontinence in women usually occurs because of problems with muscles that help to hold or release urine.

Many types of treatments are used to treat incontinence in women, depending in the severity of their problem, such as exercises, electrical stimulation, biofeedback timed voiding or bladder training, medications, pessaries, implants, surgery and catherization.

### SUMMARY OF INVENTION

It is a feature of the present invention to provide a surgically implantable urethra pressure control adjustable valve which substantially overcomes the above-mentioned disadvantages of the prior art.

Another feature of the present invention is to provide a surgically implantable urethra pressure control adjustable valve which is easy to install and provides visibility to the surgeon when positioning the valve and its pressure control means against the urethra.

Another feature of the present invention is to provide a surgically implantable urethra pressure control adjustable sphincter valve, the closing pressure of which is adjustable by injecting fluid into a balloon retained in the valve through a conduit provided with a port connector located in the scrotum or elsewhere.

Another feature of the present invention is to provide a surgically implantable urethra pressure control valve which is remotely operated by the use of a remote control device.

Another feature of the present invention is to provide a surgically implantable urethra pressure control sphincter valve and wherein the clamp of the valve is provided by spaced-apart clamping formations which are formed by rigid wire-like members adapted to pinch the urethra.

Another feature of the present invention is to provide a surgically implantable urethra pressure control valve and wherein at least the operating parts of the sphincter valve are secured in a sealed implantable housing.

According to the above features, from a broad aspect, the present invention provides a surgically implantable urethra pressure control valve comprising a clamp having a pair of spaced apart clamping arms, a urethra passage defined between said clamping arms, at least one of said clamping arms being a displaceable clamping arm movable towards the other clamping arm to a predetermined position spaced from said other clamping arm a distance sufficient to pinch and close an inner passage of said urethra when disposed in said urethra passage to arrest the flow of urine, and actuating means to effect the displacement of said displaceable clamping arm.

According to a still further broad aspect of the present invention there is provided a surgically implantable urethra pressure control sphincter valve comprising a clamp having a pair of spaced-apart clamping formations, a urethra passage defined between said clamping formations, one of said clamping formations being adapted to receive and position an inflatable balloon section of an implantable sphincter on one side of said urethra passage, said implantable sphincter having a flexile hydraulic tubing adapted to conduct hydraulic fluid sealingly injectable therein to inflate said balloon, the other of said clamping formations forming an abutment on an opposed side of said urethra passage whereby said urethra, when positioned in said urethra passage, will be pinched and closed between said balloon section when inflated and said abutment, said balloon being inflated to exert a predetermined pressure on said urethra to prevent the flow of urine from the bladder below said predetermined pressure.

### BRIEF DESCRIPTION OF DRAWINGS

A preferred embodiment of the present invention will now be described with reference to the accompanying drawings in which:

FIG. 1 is a partly fragmented side view of a first embodiment of a surgically implantable urethra pressure control valve constructed in accordance with the present invention;

FIG. 2A is a front view of FIG. 1;

FIG. 2B is a side view similar to FIG. 1 but non-fragmented;

FIG. 2C is a front view similar to FIG. 2A but showing a urethra being compressed by an actuable clamping arm;

FIG. 2D is a front view similar to FIG. 2C, but showing the urethra completely compressed by the displaceable clamping arm;

FIG. 3 is a front view of the remote controller;

FIG. 4 is a front view of a further embodiment of the surgically implantable urethra pressure control sphincter valve of the present invention, partly fragmented to show the actuating motor;

FIG. 5 is a front view showing a urethra being compressed by the displaceable clamping arm actuated by the motor;

FIG. 6 is a perspective view of a still further embodiment of the surgically implantable urethra pressure control sphincter valve of the present invention by the use resilient wire-like clamping formations;

FIG. 7A is an enlarged view of the wire clamp of FIG. 6;

FIGS. 7B and 7C are perspective views showing the clamp being closed by the expansion of a sphincter controlled balloon;

FIGS. 8A, 8B and 8C are perspective views showing a still further embodiment of the surgically implantable urethra pressure control sphincter valve using a wire-like clamp having a different construction than that of FIG. 6 and utilizing a sphincter controlled balloon as the actuating means;

FIG. 8D is a top view of FIG. 8C showing the positioning of the urethra with respect to the balloon and the clamp;

FIGS. 9A to 9C are perspective views illustrating a still further embodiment of the surgically implantable urethra pressure control sphincter valve of the present invention formed by the use of a wire-like clamping member and a sphincter balloon to close the urethra; and

FIG. 9D is a perspective view of the wire-like clamp.

### DESCRIPTION OF PREFERRED EMBODIMENTS

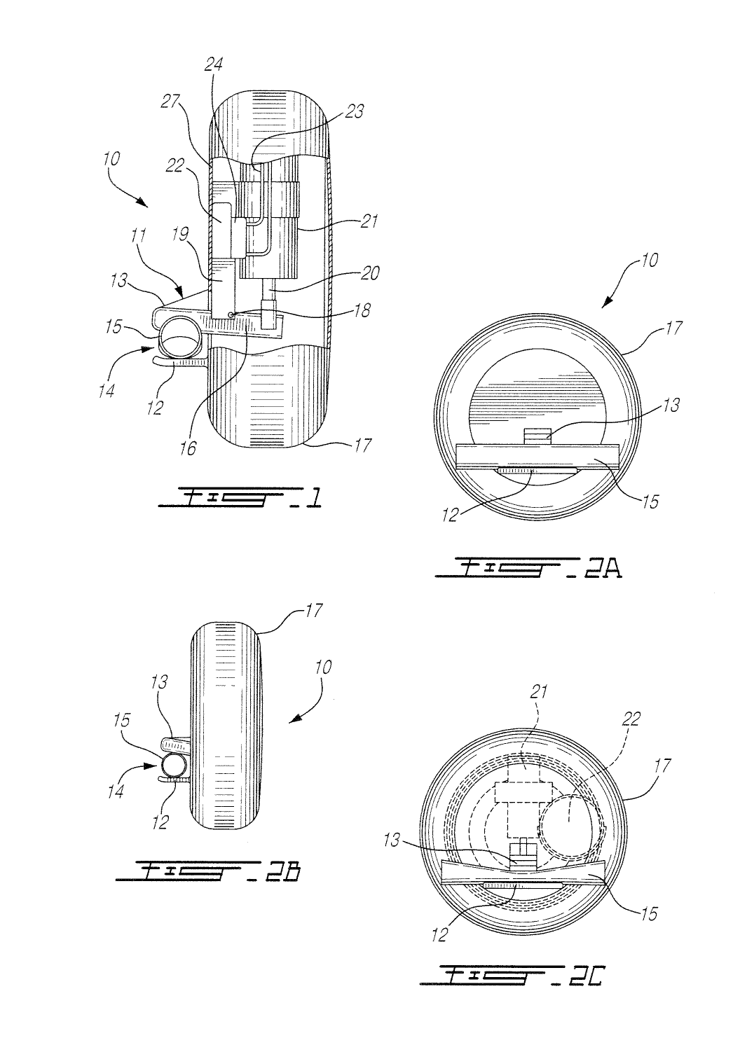

Referring now to the drawings, and more particularly to FIGS. 1 to 3, there will be described the construction and operation of a first embodiment of the surgically implantable urethra pressure control valve 10. The valve 10 comprises a clamp 11 formed by a pair of spaced-apart clamping arms, namely a stationary arm 12 and a displaceable arm 13. A urethra passage 14 is defined between the clamping arms 12 and 13. The urethra passage is dimensioned whereby to position therein the urethra 15 of a person in which the valve 10 is surgically implanted.

The displaceable arm 13 has a connecting arm 16 which extends within a sealed implantable housing 17 formed of material suitable for implanting into the body of a person and is actuable on a pivot connection 18 of a stationary member 19 by an actuable solenoid rod 20 of a battery-operated solenoid 12. A dc power cell 22 or battery operates the solenoid through electrical connections 23 and through a switch 24.

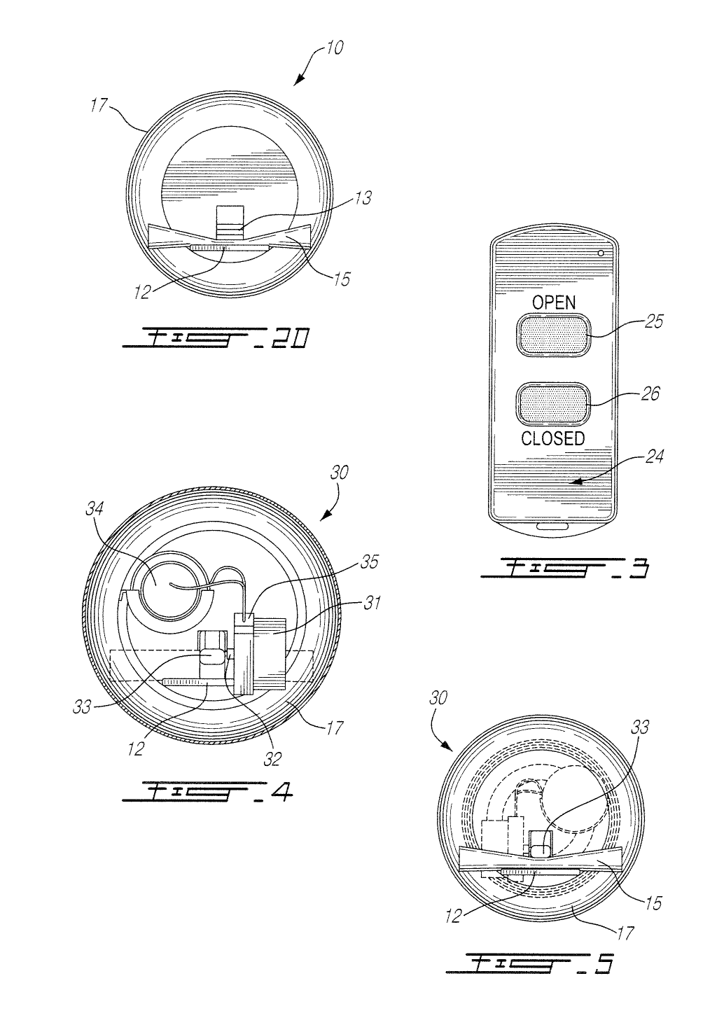

As shown in FIGS. 1 to 2B, the clamp is in a normally open condition. This clamp is remotely operated by a remote-controlled hand-held device 24, as shown in FIG. 3, which controls the switch 24. By depressing the switch button 25, the displaceable arm 13 is moved to its open position as shown in FIGS. 1 to 2B. When the closed button 26 is depressed by the wearer of the device, the solenoid is operated thereby displacing the displaceable arm 13 to move towards the stationary arm 12 thus pinching a urethra 15 positioned in the urethra passage 14 in a fashion as illustrated by FIGS. 2C and 2D. Accordingly, when the wearer person has an urge to release urine from the bladder, he simply operates the remote controller to open the urethra and discharge the bladder. Although not shown in FIG. 1, a suitable receiver circuit 27, details of which are not shown, is provided to receive the signal from the remote controller to operate the switch whereby to connect power to the solenoid.

Referring now to FIGS. 4 and 5, there will be described a still further embodiment of the surgically implantable urethra pressure control valve 30 of the present invention. As hereinshown, the actuating means is a battery-operated electric motor 31 which is immovably secured within the housing 17. The motor has an actuable shaft 32 which is coupled to the displaceable clamping arm 33 to displace the clamping arm from a urethra “open” position to a urethra “close” position, as previously described. A power cell or battery 34 operates the electric motor 31 through a switch 35. Again, a suitable receiver circuit is provided to receive the command signal from the remote controller 24 as shown in FIG. 3. As shown in FIG. 5, the clamping arm 33 is coupled to the actuating shaft 32 of the electric motor and is displaced in a similar fashion as described with respect to the embodiment of FIGS. 1 to 2D.

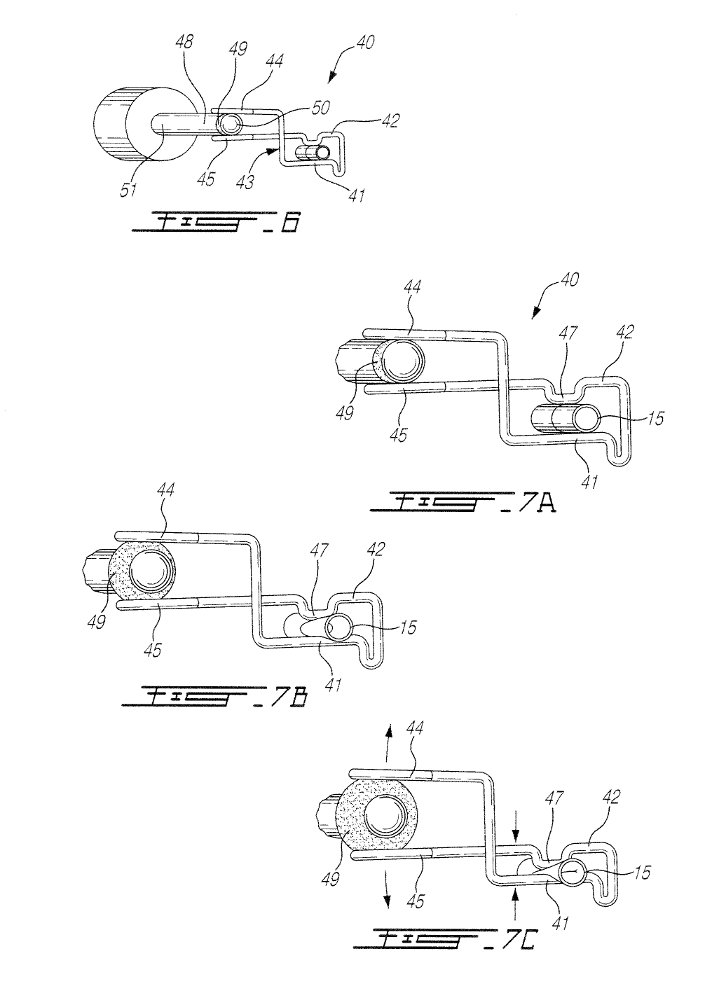

Referring now to FIGS. 6 to 7C, there will be described another embodiment of the surgically implantable urethra pressure control valve 40 of the present invention incorporating a control sphincter. As hereinshown, the valve is constructed by a wire-like formation formed of a rigid spring stainless steel wire or suitable clinically approved rigid plastics material having a memory capable of retaining its shape and defining a pair of spaced-apart clamping arms and 42. The shaped wire member 43 further defines actuating formations 44 and 45 in a free end of the wire-like clamp 43. These actuating formations are circular loops formed in the spaced free ends of the wire-like clamp formation 43.

A urethra passage 46 is defined between the clamping arms 41 and 42. As shown more clearly in FIG. 7A, the clamping arm 42 has an inner projecting pinching formation 47 which is hereinshown as a narrow projecting formation but it could be much wider than that as hereinshown. The urethra passage 46 is defined under this pinching formation 47. FIGS. 6 and 7A show the clamp in a normal position. Although not shown, this clamp may be housed in a surgically implantable pouch having passages to receive the urethra 15 therethrough and a free end of an inflatable balloon section 48 of an implantable sphincter having an inflatable balloon 49 adjacent a free end 50 of section 48 with the balloon 49 being located between the loops of the actuating formations 44 and 45 and this is more clearly shown in FIGS. 7B and 7C. The implantable sphincter has a flexible hydraulic tube 51 to conduct hydraulic fluid which is sealingly injectable therein by a syringe to inflate the balloon 49 to a controlled size as shown in FIG. 7C. As shown in FIGS. 7B and 7C, by inflating the balloon to a controlled size, the actuating formations 44 and 45 are caused to move away from one another thereby causing the clamping arms 41 and 42 to move closer to one another to effect the pinching and closing of the urethra 15 positioned in the urethra passage 46. This pinching pressure is predetermined whereby the urethra can open upon pressure built-up by the urine accumulation in the bladder causing the user to exercise a need to evacuate urine.

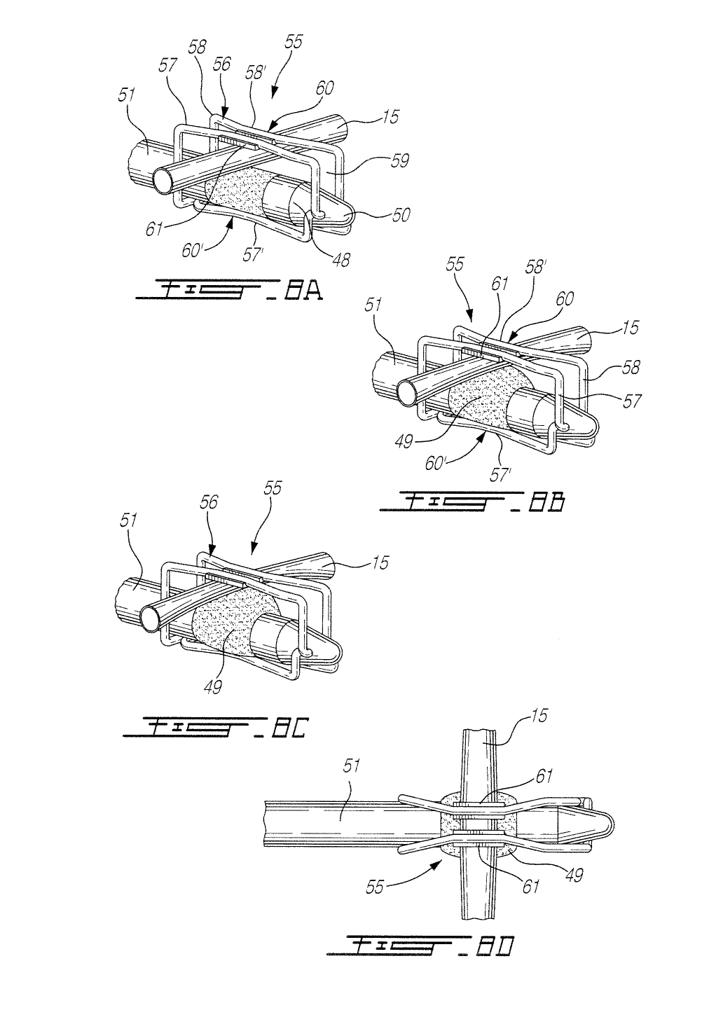

Referring now to FIGS. 8A to 8D, there will be described a still further embodiment of the surgically implantable urethra pressure control sphincter valve 55 of the present invention. As hereinshown the clamp is constituted by a rigid wire-like member 56 constructed with suitable material as previously described, and shaped to define spaced-apart clamping formations. The wire-like member 56 defines a cage formed by opposed parallel rectangular restraining side formations 57 and 58 which define therebetween a restricted longitudinal open-ended passage 59 to receive and retain the free end section 48 of the sphincter as previously described with respect to FIG. 6 and including the inflatable balloon. The opposed parallel rectangular restraining side formations 57 and 58 have an inwardly angled top and bottom wire section 57′ and 58′ to form top and bottom wire restraining formations 60 and 60′. The top wire restraining formation 60 constitutes an abutment against which the urethra 15 is pinched and it may have suitable cushioning members 61 secured to the wire-like members in the area of the inwardly angled portion 58′ to abut against the urethra. As shown in FIG. 8A, the free end section 48 of the sphincter containing the balloon is in its operating position.

Referring now to FIGS. 8B and 8C, there is shown the operation of the valve. As previously described, when hydraulic fluid is sealingly injected into the sphincter tubing 51, the balloon 49 inflates. Because the urethra 15 is disposed between the balloon and the abutment 60, when the balloon increases in size, it closes the urethra by pinching the urethra between the cushions 61 and the balloon 49 effecting a closure. The pressure applied is a control pressure which is sufficient to cause the pressure of the fluid in the bladder to open the urethra when the user exercises a need to evacuate fluid thereby causing the clamp section of the spring to flex or causing the balloon to deform causing an opening of the urethra sufficient to evacuate urine.

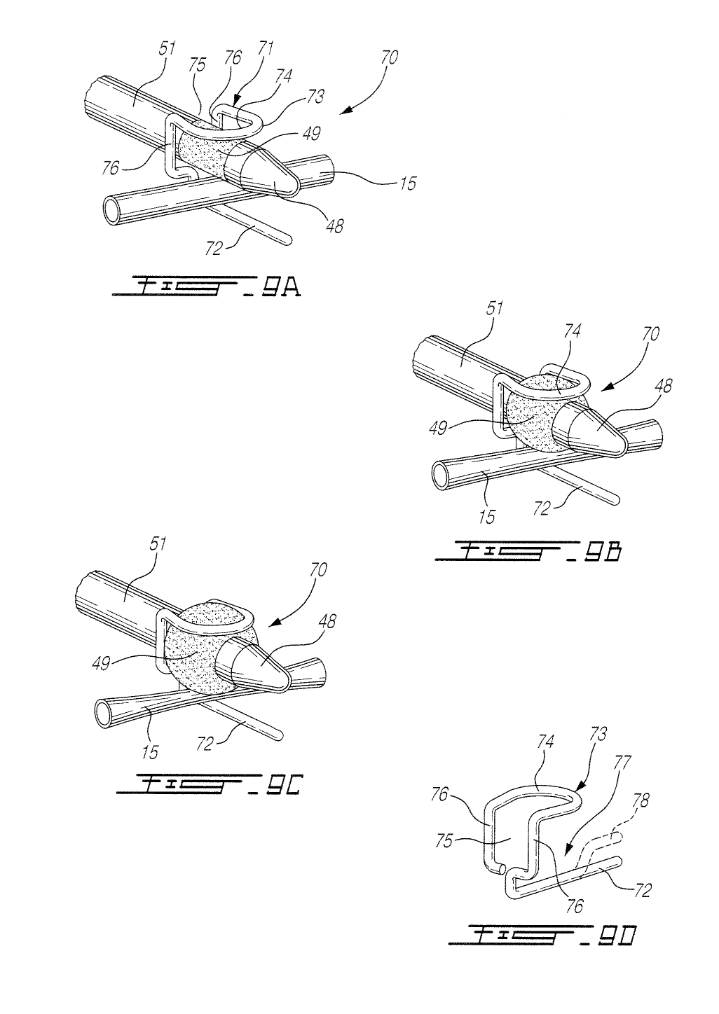

Referring now to FIGS. 9A to 9D, there is described another embodiment of the surgically implantable urethra pressure control sphincter valve 70 of the present invention. As hereinshown the clamp 71 is also formed of either stainless steel wire or a wire-like member formed from a clinically certified synthetic material capable of being molded and having a memory to retain its shape. It is also shaped to define the spaced-apart clamping formations herein constituted by a straight wire end section 72 which defines one clamping formation, and a U-shaped right-angled formation which defines a balloon nesting section 73. The balloon nesting section 73 is defined by the U-shaped wire end portion 74. The clamp 71 is also shaped whereby to define a sphincter passage 75 between opposed transverse arms 76 of the U-shaped right-angle formation. The urethra passage 77, as better shown in FIG. 9D, extends between the balloon nesting section 73 and the straight wire end section 72 and extending transversely to the straight wire section.

As shown in FIG. 9D, the straight wire end section 72 may also have an upwardly extending free end section 78 as shown in stippled lines and projecting towards the nesting section 73 whereby to better restrain the urethra 15 in position in the urethra passage 77.

As shown in FIGS. 9B and 9C, by inflating the balloon 49 of the sphincter, control pressure is applied against the urethra 15 to close the urethra. Evacuation of urine is effected in the same fashion as previously described with reference to FIGS. 6 to 8D.

It is also contemplated that the battery for the devices be accessible outside the skin of the wearer person and connected by an implanted wire. This permits for the recharging or replacement of the battery. The battery could be protected by a waterproof adhesive tape, a waste band, etc.

It is within the ambit of the present invention to cover any obvious modifications of the preferred embodiments described herein, provided such modifications fall within the scope of the appended claims.

I claim:

1. A surgically implantable urethra pressure control valve comprising a clamp having a pair of spaced apart clamping arms, a urethra passage defined between said clamping arms, at least one of said clamping arms being a displaceable clamping arm movable towards the other clamping arm to a predetermined position spaced from said other clamping arm a distance sufficient to pinch and close an inner passage of said urethra when disposed in said urethra passage to arrest the flow of urine, and actuating means to effect the displacement of said displaceable clamping arm.

2. A surgically implantable urethra pressure control valve as claimed in claim 1 wherein there is further provided a sealed implantable housing for housing said actuating means.

3. A surgically implantable urethra pressure control valve as claimed in claim 2 wherein the other of said clamping arms is a stationary urethra abutment arm immovably secured to an outer surface of said housing, said displaceable clamping arm extending from an opening in said outer surface.

4. A surgically implantable urethra pressure control valve as claimed in claim 2 wherein said actuating means is a battery-operated solenoid immovably secured in said housing, said solenoid having an actuable solenoid rod secured to a connecting arm of said displaceable clamping arm to displace said arm from a urethra open position to a urethra closed position.

5. A surgically implantable urethra pressure control valve as claimed in claim 4 wherein there is further provided receiver circuit means in said housing an a battery cell, switch means connecting said battery cell to said solenoid to operate said solenoid to open and close said urethra to evacuate urine therethrough, and a remote hand-held control device to operate said switch means.

6. A surgically implantable urethra pressure control valve as claimed in claim 2 wherein said actuating means is a battery-operated electric motor immovably secured in said housing, said motor having an actuatable shaft coupled to said displaceable clamping arm to displace said clamping arm from a urethra open position to a urethra closed position.

7. A surgically implantable urethra pressure control valve as claimed in claim 6 wherein there is further provided receiver circuit means in said housing, a battery cell, switch means connecting said battery cell to said solenoid to operate said solenoid to open and close said urethra to evacuate urine therethrough, and a remote hand-held control device to operate said switch means.

8. A surgically implantable urethra pressure control valve as claimed in claim 1 wherein said clamping arms are formed from a resilient member shaped to define said pair of spaced-apart clamping arms, said actuating means being defined between opposed end formations of said resilient member and constituted by an inflatable balloon section of an implantable sphincter having a flexible hydraulic tubing adapted to conduct hydraulic fluid sealingly injectable therein to inflate said balloon to a controlled size whereby to cause said resilient member to flex and said clamping arms to move closer to one another to effect said pinching and closing said inner passage of said urethra.

9. A surgically implantable urethra pressure control valve comprising a clamp having a pair of spaced-apart clamping formations, a urethra passage defined between said clamping formations, one of said clamping formations being adapted to receive and position an inflatable balloon section of an implantable sphincter on one side of said urethra passage, said implantable sphincter having a flexile hydraulic tubing adapted to conduct hydraulic fluid sealingly injectable therein to inflate said balloon, the other of said clamping formations forming an abutment on an opposed side of said urethra passage whereby said urethra, when positioned in said urethra passage, will be pinched and closed between said balloon section when inflated and said abutment, said balloon being inflated to exert a predetermined pressure on said urethra to prevent the flow of urine from the bladder below said predetermined pressure.

10. A surgically implantable urethra pressure control valve as claimed in claim 9 wherein said clamp is constituted by a rigid wire-like member shaped to define said spaced-apart clamping formations, said wire-like member defining opposed parallel rectangular restraining side formations defining a restricted formation defining a balloon nesting section at a U-shaped wire end portion and a sphincter passage between transverse opposed arms of said U-shaped right angle formation, said urethra passage extending between said balloon nesting section and said straight wire end section and transverse thereto.

11. A surgically implantable urethra pressure control valve as claimed in claim 9 wherein said clamp is constituted by a rigid wire-like member shaped to define said spaced-apart clamping formations, said wire-like member having a straight wire end section defining one clamping formation, and a U-shaped right-angled formation defining a balloon nesting section at a U-shaped wire end portion and a sphincter passage between transverse opposed arms of said U-shaped right angle formation, said urethra passage extending between said balloon nesting section and said straight wire end section and transverse thereto.

12. A surgically implantable urethra pressure control valve as claimed in claim 11 wherein said straight wire end section ahs an upwardly extending end section projecting towards said nesting section to define a restraining abutment for a urethra disposed in said urethra passage.

13. A surgically implantable urethra pressure control valve as claimed in claim 10 wherein said wire-like member is one of a stainless steel wire or a wire-like member formed from a clinically certified synthetic material capable of being molded and having a memory to retain its shape.

14. A surgically implantable urethra pressure control valve as claimed in claim 11 wherein said wire-like member is one of a stainless steel wire or a wire-like member formed from a clinically certified synthetic material capable of being molded and having a memory to retain its shape.

15. A surgically implantable urethra pressure control valve as claimed in claim 12 wherein said wire-like member is one of a stainless steel wire or a wire-like member formed from a clinically certified synthetic material capable of being molded and having a memory to retain its shape.

16. A surgically implantable urethra pressure control valve as claimed in claim 9 wherein there is further provided a sealed implantable pouch for housing at least part of said urethra pressure control valve.

17. A surgically implantable urethra pressure control valve as claimed in claim 10 wherein there is further provided a sealed implantable pouch for housing at least part of said urethra pressure control valve.

18. A surgically implantable urethra pressure control valve as claimed in claim 11 wherein there is further provided a sealed implantable pouch for housing at least part of said urethra pressure control valve.

19. A surgically implantable urethra pressure control valve as claimed in claim 12 wherein there is further provided a sealed implantable pouch for housing at least part of said urethra pressure control valve.

20. A surgically implantable urethra pressure control valve as claimed in claim 13 wherein there is further provided a sealed implantable pouch for housing at least part of said urethra pressure control valve.
